(19) United States
(12) Patent Application Publication
(10) Pub. No.: US 2025/0364074 A1
(43) Pub. Date: Nov. 27, 2025

(54) Antibody Competition Model Using Hidden Variable Affinities

(71) Applicant: AbCellera Biologics Inc., Vancouver (CA)

(72) Inventors: Christopher Thaddeus HUGHES, Vancouver (CA); Valentine Julie Layla BERTRAND DE PUYRAIMOND, Vancouver (CA); Thomas Roderick DOCKING, Vancouver (CA); Lucas KRAFT, Vancouver (CA); Stefan Edward HANNIE, Vancouver (CA); Kevin Richard JEPSON, Vancouver (CA); Tomas GOGORZA, Vancouver (CA); Jordan John YAP, Vancouver (CA); Alexander Sewall FORD, Vancouver (CA)

(73) Assignee: AbCellera Biologics Inc., Vancouver, BC (CA)

(21) Appl. No.: 17/860,760

(22) Filed: Jul. 8, 2022

Related U.S. Application Data
(60) Provisional application No. 63/219,578, filed on Jul. 8, 2021.

(51) Int. Cl. G16B 15/30; G16B 40/20

(52) U.S. Cl. CPC G16B 15/30; G16B 40/20

(57) ABSTRACT

Embodiments derive hidden variables based on antibody competition data to discover binding patterns. For example, antibody competition data for a plurality of antibodies and an antigen can be received, where the antibody competition data includes data values indicative of pairwise competition between antibodies. The antibody competition data can be processed to generate training data. Using the training data and an optimization engine, a plurality of hidden variables and affinity scores for the hidden variables can be derived, where affinity scores for the hidden variables are derived for each antibody and the hidden variables represent competition factors for the antigen that cause competition among the antibodies.

### STATEMENT REGARDING FEDERALLY SPONSORED RESEARCH OR DEVELOPMENT

This invention was made with U.S. Government support under D18AC00002 awarded by the Defense Advanced Research Projects Agency. The U.S. Government has certain rights in the invention.

### FIELD

The embodiments of the present disclosure generally relate to deriving hidden variables based on antibody competition data to discover binding patterns.

### BACKGROUND

Monoclonal antibody (“mAB”) discovery is a complex, time consuming, and resource intensive technological challenge. One component of mAB discovery involves understanding how antibodies compete when binding to an antigen. Epitope binning is an informative technique that can further this understanding. However, conventional approaches achieve models with only a limited view of how these antibodies compete.

### SUMMARY

The embodiments of the present disclosure are directed to systems and methods for deriving hidden variables based on antibody competition data to discover binding patterns. Antibody competition data for a plurality of antibodies and an antigen can be received, where the antibody competition data includes data values indicative of pairwise competition between antibodies. The antibody competition data can be processed to generate training data. Using the training data and an optimization engine, a plurality of hidden variables and affinity scores for the hidden variables can be derived, where affinity scores for the hidden variables are derived for each antibody and the hidden variables represent competition factors for the antigen that cause competition among the antibodies.

Features and advantages of the embodiments are set forth in the description which follows, or will be apparent from the description, or may be learned by practice of the disclosure.

### BRIEF DESCRIPTION OF THE DRAWINGS

Further embodiments, details, advantages, and modifications will become apparent from the following detailed description of the preferred embodiments, which is to be taken in conjunction with the accompanying drawings.

FIG. 1 illustrates a system for deriving hidden variables based on antibody competition data to discover binding patterns according to an example embodiment.

FIG. 2 illustrates a diagram of a computing system according to an example embodiment.

FIG. 3 illustrates a conventional heatmap that indicates competition data for monoclonal antibodies.

FIG. 4 illustrates a previous network approach for binning monoclonal antibodies based on competition data.

FIG. 5 illustrates a competition dynamic for monoclonal antibodies.

FIG. 6 illustrates a flowchart for deriving hidden variables based on antibody competition data to discover binding patterns according to an example embodiment.

### DETAILED DESCRIPTION

Embodiments derive hidden variable information indicative of competition patterns among monoclonal antibodies based on pairwise antibody competition data. For example, a predictive mathematical model of antibody-antigen binding can be discovered by an optimization engine. In some embodiments, the optimization engine can derive a set of hidden variables that form the foundation for generating predictions about whether a pair of antibodies will compete with each other. These hidden variables can be loosely thought of as the epitope binding resources or “antigen real estate” that are used by the antibody when binding.

In some embodiments, the variables are “hidden” because the model is agnostic about where these resources actually exist on the antigen surface. For example, each hidden variable can be a placeholder for some epitope resource on the antigen that an antibody uses to bind. In some implementations, some hidden variables can also represent some other competition factor (e.g., other than epitope/location competition).

In some embodiments, the optimization engine can generate hidden variable logit values and compare these logit values to observed competition data values (e.g., pairwise antibody competition) present in the training data for the antibodies. In some embodiments, a loss function can be optimized by implementing a gradient that adjusts the antibodies' hidden variable logit values until the loss function is optimized and/or a metric is achieved (e.g., convergence is achieved). For example, the optimization of hidden variable logit values for an antibody can achieve hidden variable affinity scores that indicate/predict the antibody's level of competition for the competition factor represented by the hidden variable (e.g., for the epitope on the antigen represented by the hidden variable). In some embodiments, pairwise competition prediction scores between antibodies can be generated using the logit values for the antibodies.

Embodiments can also implement ensemble learning techniques by combining predictions (e.g., competition scores) from multiple hidden variable models trained on different antibody competition data. For example, each hidden variable model can be trained using competition data for different sets of antibodies. A prediction about whether two antibodies would compete can be generated by combining the competition scores from several trained hidden variable models.

In some embodiments, a landmark antibody correlation model can use competition measurements for a predetermined set of landmark antibodies to predict pairwise competition (e.g., against a particular antigen) between antibodies that have not been measured. For example, given a pair of antibodies for which competition predictions are desired, a correlation can be calculated between each antibody's competition measurements with the landmark antibodies. Based on the correlation value, a competition likelihood can be predicted.

Conventional epitope binning involves the testing of antibodies (e.g., using a device that performs an “experimental run”) in a combinatorial manner (e.g., pairwise) to derive competition data that is analyzed so that antibodies that compete for the same binding region (e.g., epitope) are grouped together into bins. Epitope binning experiments generate large amounts of data. For example, in some binning experiments, for each experimental run a data point (e.g., numeric value) is generated for every pair of participating antibodies. Some runs can include up to 384 antibodies per experiment, which means there would be up to 384*384=147,456 observations about the pairwise competition between different antibodies. Furthermore, at times it would be advantageous to perform epitope binning across even larger groups of antibodies than current devices support in a single experimental run, or it would be advantageous to extend a prior epitope binning run with newly discovered antibodies without running competition experiments on all pairs of these antibodies.

Embodiments achieve improved model(s) for analyzing and understanding the results of a single or multiple epitope binning runs. Further, the improved model(s) can attribute experimental outcomes to properties of individual antibodies such that they can be grouped together in more informative ways than just assigning each antibody to a single bin. Embodiments support techniques to combine the results from multiple epitope binning experiments, which are limited by current device limitations to 384 antibodies at one time. Embodiments can also extend an existing epitope binning run with new antibodies without repeating the entire experiment. In addition, for antibodies that participated in different epitope binning runs (e.g., for which there is no direct experimental information about whether they compete) embodiments of the model(s) support predictions about whether or not these antibodies will compete.

Embodiments optimize techniques to collect and organize pairwise antibody competition measurements against a particular antigen by using model(s) that can predict those pairwise antibody competition measurements prior to (or without) performing experimental runs to actually measure them. Accordingly, embodiments can significantly reduce the number of experimental runs necessary to generate desired antibody competition data (and significantly improve resource efficiency) when compared to conventional epitope binning approaches.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

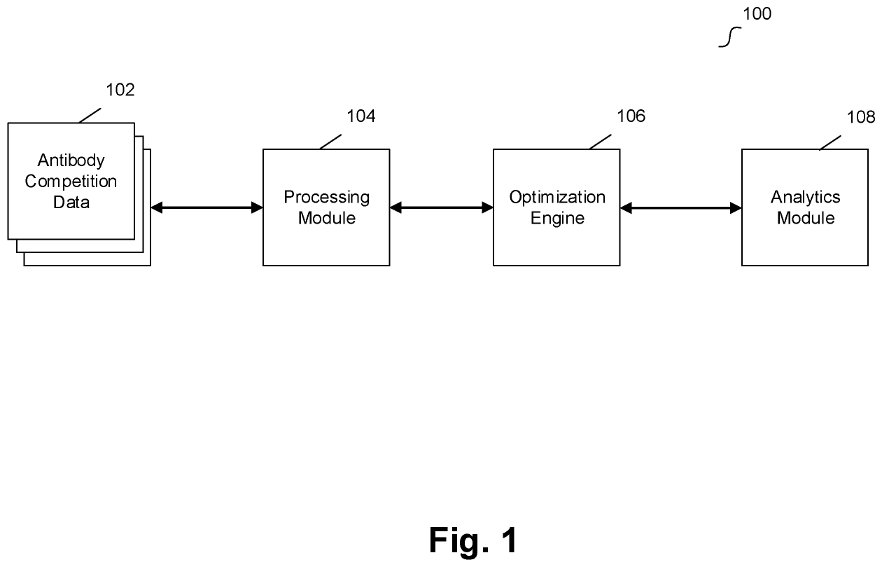

FIG. 1 illustrates a system for deriving hidden variables based on antibody competition data to discover binding patterns according to an example embodiment. System 100 includes antibody competition data 102, processing module 104, optimization engine 106, and analytics module 108. For example, antibody competition data 102 can include data generated from surface plasmon resonance (“SPR”) experimental techniques that generate numerical results characterizing antibodies and their interactions (e.g., pairwise competition) with an antigen. In some implementations, antibody competition data is generated using a Carterra® LSA™ instrument.

In some embodiments, antibody competition data 102 can include data from several experimental runs. For example, an experimental run can generate numerical values that indicate pairwise competition between two antibodies for a given antigen, and in total antibody competition data 102 can include data for interactions between several (e.g., tens, hundreds, or thousands) of antibodies. In some embodiments, competition data 102 comprises binary pairwise competition data that indicates whether two antibodies compete using a binary value (e.g., 1 or 0, true or false, and the like)

Processing module 104 can process antibody competition data 102 such that training data is generated for optimization engine 106. For example, antibody competition data 102 can include data from multiple experimental runs, and processing module 104 can combine this data in a manner suitable for processing by optimization engine 106. Embodiments of processing module 104 can also transform numerical values from competition data 102 using a function (e.g., a function that assigns a binary value), or perform other suitable data transformations.

Optimization engine 106 can derive hidden variables and hidden variable affinity scores for participating antibodies based on the training data generated by processing module 104. For example, optimization engine 106 can generate hidden variable logit values (e.g., logit values that represent the antibodies' hidden variable affinity scores) and compare these logit values to observed competition data values (e.g., pairwise antibody competition) present in the training data for the antibodies. In some embodiments, a loss function can be optimized by implementing a gradient that adjusts the antibodies' hidden variable logit values until the loss function is optimized and/or a metric is achieved (e.g., convergence is achieved).

For example, the optimization of hidden variable logit values for an antibody can achieve hidden variable affinity scores that indicate/predict the antibody's level of competition for the competition factor represented by the hidden variable (e.g., for the epitope on the antigen represented by the hidden variable). In some implementations, the hidden variables may correlate to competition factors for antigen binding beyond epitope location (e.g., interfering/competing factors beyond competing for the same binding location).

Analytics module 108 can generate competition information for antibodies based on the output from optimization engine 106. For example, optimization engine 106 can output a model for predicting/discovering competition among a plurality of antibodies. In particular, the model generated by optimization engine 106 may discover antibodies that compete over different competition factors (e.g., different epitopes or other competing factors). Accordingly, analytics module 108 can be used to generate a panel of antibodies with differing hidden variable affinity values (e.g., antibodies that compete over the antigen in different ways). Such a panel can offer a diversity of pathways to positive treatment outcomes, and thus represents an improvement to manufacturing/discovering monoclonal antibodies that deliver positive health outcomes.

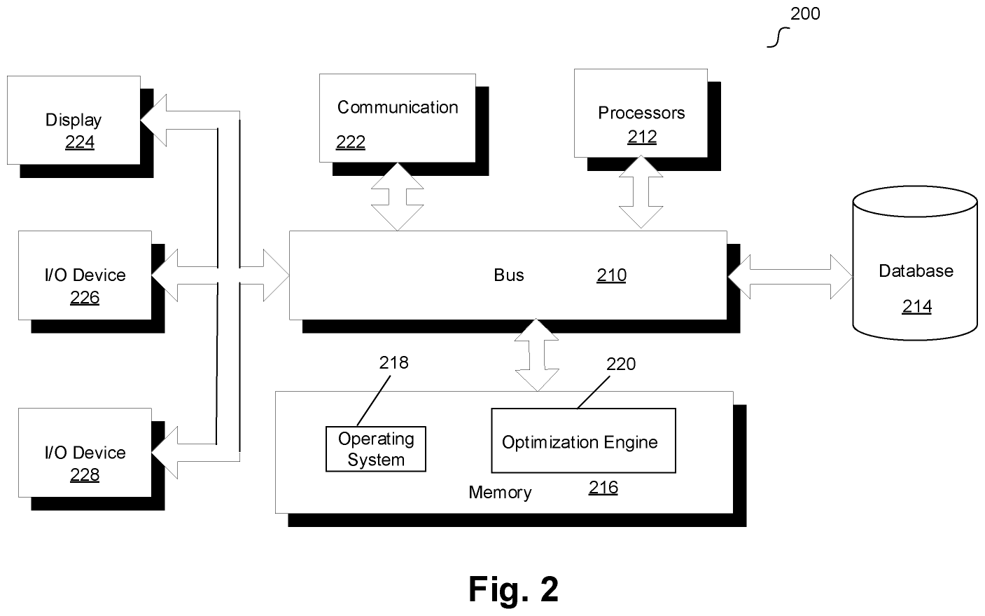

FIG. 2 is a diagram of a computing system 200 in accordance with embodiments. As shown in FIG. 2, system 200 may include a bus 210, as well as other elements, configured to communicate information among processor 212, data 214, memory 216, and/or other components of system 200. Processor 212 may include one or more general or specific purpose processors configured to execute commands, perform computation, and/or control functions of system 210. Processor 212 may include a single integrated circuit, such as a micro-processing device, or may include multiple integrated circuit devices and/or circuit boards working in combination. Processor 212 may execute software, such as operating system 218, optimization engine 220, and/or other applications stored at memory 216.

Communication component 222 may enable connectivity between the components of system 200 and other devices, such as by processing (e.g., encoding) data to be sent from one or more components of system 200 to another device over a network (not shown) and processing (e.g., decoding) data received from another system over the network for one or more components of system 200. For example, communication component 222 may include a network interface card that is configured to provide wireless network communications. Any suitable wireless communication protocols or techniques may be implemented by communication component 222, such as Wi-Fi, Bluetooth®, Zigbee, radio, infrared, and/or cellular communication technologies and protocols. In some embodiments, communication component 222 may provide wired network connections, techniques, and protocols, such as an Ethernet.

System 200 includes memory 216, which can store information and instructions for processor 212. Embodiments of memory 216 contain components for retrieving, reading, writing, modifying, and storing data. Memory 216 may store software that performs functions when executed by processor 212. For example, operating system 218 (and processor 212) can provide operating system functionality for system 200. Optimization engine 220 (and processor 212) can generate a model for predicting/discovering antibody competition according to embodiments. Embodiments of optimization engine 220 may be implemented as an in-memory configuration. Software modules of memory 216 can include operating system 218, optimization engine 220, as well as other applications modules (not depicted).

Memory 216 includes non-transitory computer-readable media accessible by the components of system 200. For example, memory 216 may include any combination of random access memory (“RAM”), dynamic RAM (“DRAM”), static RAM (“SRAM”), read only memory (“ROM”), flash memory, cache memory, and/or any other type of non-transitory computer-readable medium. A database 214 is communicatively connected to other components of system 200 (such as via bus 210) to provide storage for the components of system 200. Embodiments of database 214 can store data in an integrated collection of logically-related records or files.

Database 214 can be a data warehouse, a distributed database, a cloud database, a secure database, an analytical database, a production database, a non-production database, an end-user database, a remote database, an in-memory database, a real-time database, a relational database, an object-oriented database, a hierarchical database, a multi-dimensional database, a Hadoop Distributed File System (“HFDS”), a NoSQL database, or any other database known in the art. Components of system 200 are further coupled (e.g., via bus 210) to: display 224 such that processor 212 can display information, data, and any other suitable display to a user, I/O device 226, such as a keyboard, and I/O device 228 such as a computer mouse or any other suitable I/O device.

In some embodiments, system 200 can be an element of a system architecture, distributed system, or other suitable system. For example, system 200 can include one or more additional functional modules, which may include the various modules of a Carterra® LSA™ instrument, any other suitable device for generating antibody competition data, or any other suitable modules.

Embodiments of system 200 can remotely provide the relevant functionality for a separate device. In some embodiments, one or more components of system 200 may not be implemented. For example, system 200 may be a tablet, smartphone, or other wireless device that includes a display, one or more processors, and memory, but that does not include one or more other components of system 200 shown in FIG. 2. In some embodiments, implementations of system 200 can include additional components not shown in FIG. 2. While FIG. 2 depicts system 200 as a single system, the functionality of system 200 may be implemented at different locations, as a distributed system, within a cloud infrastructure, or in any other suitable manner. In some embodiments, memory 216, processor 212, and/or database 214 are be distributed (across multiple devices or computers that represent system 200). In one embodiment, system 200 may be part of a computing device (e.g., smartphone, tablet, computer, and the like).

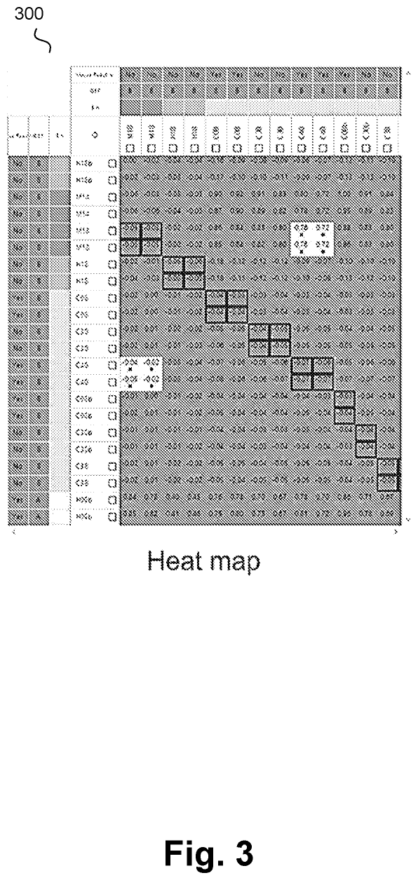

Monoclonal antibody (“mAB”) discovery is a complex, time consuming, and resource intensive technological challenge. One component of mAB discovery involves understanding how antibodies compete when binding to an antigen. Epitope binning is an informative approach to further this understanding. In particular, conventional epitope binning involves the testing of antibodies (e.g., using a device that performs an “experimental run”) in a combinatorial manner (e.g., pairwise) to derive competition data that is analyzed so that antibodies that compete for the same binding region (e.g., epitope) are grouped together into bins. Example competition data generated by an experimental run is depicted in heatmap 300 of FIG. 3. Heatmap 300 comprises different antibodies across the rows and columns, where the numeric values at the intersection of two antibodies indicates the pairwise competition between them.

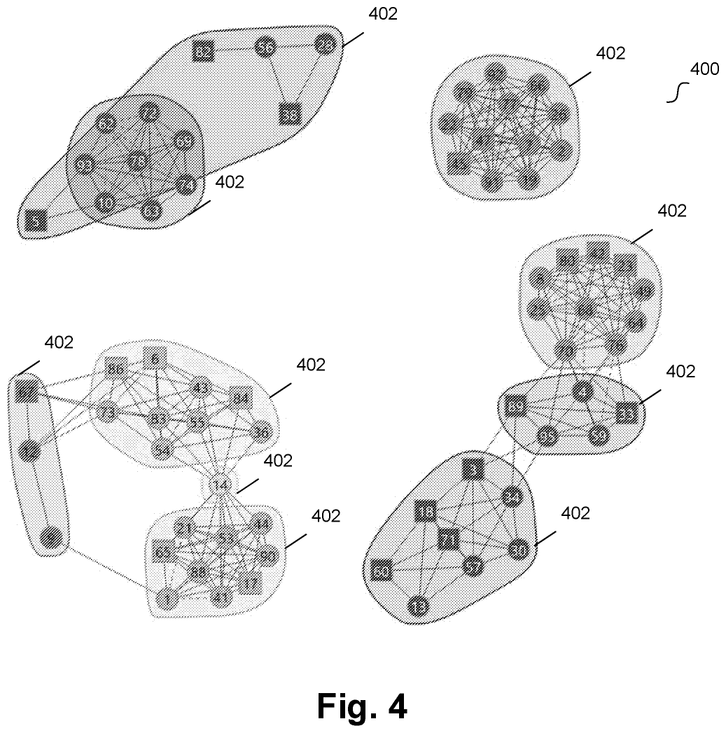

Some prior approaches to epitope binning are based on graph clustering algorithms. In these approaches, an antibody is assigned into a single cluster based on proximity or number of connecting edges within the competition graph. FIG. 4 illustrates a conventional network approach for binning monoclonal antibodies based on competition data. Network graph 400 includes bins 402 that use a prior graph clustering approach. As depicted in FIG. 4, each antibody is assigned a single bin 402, or cluster, based on the antibody's competition profile.

Rather than assigning an antibody a single bin, embodiments assign each antibody numeric affinity scores based on a set of hidden variables. For example, these hidden variables can be used to predict competition between pairs of antibodies that were not observed, and are also inherently useful in understanding the binding patterns an antibody uses to attach to an antigen.

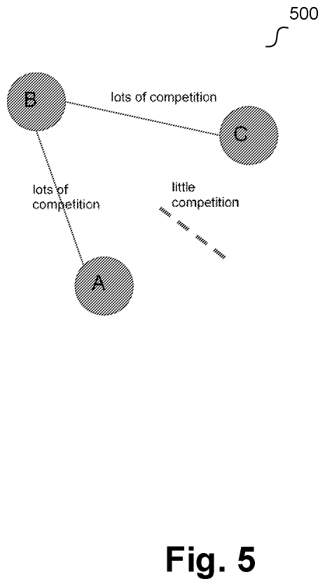

Several benefits are achieved by the hidden variable approach implemented by embodiments. For example, embodiments can model observed experimental data with higher fidelity than a cluster-based model that assigns each antibody into a single cluster. Specifically, if the experimental data shows a non-transitive pattern of antibody competition, this cannot be well represented using a model that assigns antibodies to a single cluster. FIG. 5 illustrates a competition dynamic for monoclonal antibodies that illustrates this flaw in previous approaches. Concretely, diagram 500 depicts that:
Group A competes a substantial amount with Group B
Group B competes a substantial amount with Group C
Group A DOES NOT compete a substantial amount with Group C

A cluster-based model cannot decide which single cluster these groups should be assigned. However, the “hidden variable” model can explain this pattern of competition by assigning antibodies in each group different affinities to two different hidden variables:

| | Hidden Variable 0 | Hidden Variable 1 |
|---|---|---|
| Group A | High affinity | Low affinity |
| Group B | High affinity | High affinity |
| Group C | Low affinity | High affinity |

Accordingly, while previous approaches had limited insight, higher fidelity analytics can be derived using embodiments of the hidden variable approach. It may be useful to consider the hidden variables as enabling a single antibody to belong to more than one cluster at a time, and with a numeric affinity rather than a binary judgement about membership to a particular group. Together, these properties enable the model(s) to make predictions about antibody competition.

Another limitation of a cluster-based approach is that the resultant model cannot make robust predictions about whether antibodies compete, such as by combining competition data to generate a larger competition matrix across different runs of the experimental equipment. Embodiments generate model(s) that assigns numeric affinities for each antibody to different hidden variable rather than just assigning each antibody into a single cluster. This approach supports numeric predictions about whether antibodies from different epitope binning runs will compete with each other.

The advantage of joining together data from multiple epitope binning runs can be thought of as a novel approach to the commonly known matrix completion problem. For example, often multiple epitope binning runs do not fully intersect, so the matrix describing all pairs of antibody competition is incomplete (e.g., if antibodies spanning multiple runs are listed on rows and column in tabular form, data for some of the intersections will be missing). In the case where the matrix represents pairwise antibody competition, the hidden variable affinity approach taken by embodiments can “complete” the incomplete matrix by way of optimization based on the available competition data.

In some embodiments, the numeric predictions coming from the model(s) can be interpreted as a confidence score, which allows the model(s) to incorporate noisy and/or conflicting experimental evidence and thus make predictions with higher or lower confidence (e.g., depending on the strength of the evidence). An additional benefit of the hidden variable affinity scores is that the model(s) support dimensionality reduction techniques, such as t-Distributed Stochastic Neighbor Embedding (“t-SNE”) or Uniform Manifold Approximation and Projection (“UMAP”), so that two-dimensional clustering plots showing the relationships between groups of antibodies spanning multiple epitope binning runs can be generated. For example, the pairwise distance matrix can be computed, using any suitable distance metric such as Euclidean, Manhattan, and the like, between the hidden variable affinities for each antibody, and that distance matrix can be run through a dimensionality reduction system such as UMAP. Some techniques can also impute a full competition matrix for a set of epitope binning runs, compute a pairwise distance matrix for the antibodies using the distances between their columns and rows, and send that pairwise distance matrix through a dimensionality reduction system.

The hidden variable affinity score in embodiments can be stored as a table of “hidden logits” that represent the affinity of each antibody with each hidden variable. For example, the hidden logits can be any finite number. In some embodiments, positive values represent higher affinities and negative values represent lower affinities. Below is an example table showing each antibody's hidden logit value, for a set of antibodies and 3 hidden variables.

| | H0 logit | H1 logit | H2 logit |
|---|---|---|---|
| Ab1 | 2.20 | −2.20 | −0.85 |
| Ab2 | 1.39 | −2.20 | 0.00 |
| Ab3 | −2.20 | 2.20 | −1.39 |
| Ab4 | 0.85 | 1.39 | −2.20 |

Prior to using the hidden logits in the model, the numeric values can be sent through the sigmoid function in some embodiments. For example, the sigmoid function can transform them into the range of (0 . . . 1), where hidden logit values greater than zero become hidden variables greater than 0.5. Below is an example of the hidden logits

| | H0 | H1 | H2 |
|---|---|---|---|
| Ab1 | 0.9 | 0.1 | 0.3 |
| Ab2 | 0.8 | 0.1 | 0.5 |
| Ab3 | 0.1 | 0.9 | 0.2 |
| Ab4 | 0.7 | 0.8 | 0.1 |

The label “hidden logits” is used because these values represent the normalized affinity score between each antibody and each hidden variable. In some embodiments, model fitting is used to saturate the hidden variable affinities as close to 0 or 1 as possible so that they can be interpreted as binary judgements about whether an antibody requires a particular hidden variable to bind, although that is not always possible due to conflicting evidence and other factors.

In some embodiments, a prediction about whether two antibodies would compete is based on a measure of how much these two antibodies require overlapping hidden variable resources. One approach to accomplish this measure is the dot-product operation, multiplying together the values in corresponding columns within the rows in question. Finally, the predicted competition score is sent through a sigmoid operation in some embodiments, so that that the values are within the range of (0 . . . 1).

Below is an example algorithm that demonstrates how the model predicts whether two antibodies compete according to some embodiments:

$$competes ( {Ab}_{1} , {Ab}_{2} ) = sigmoid ( - α + 2 α {\sum }_{h = 0}^{N - 1} HV [ {Ab}_{1} , {H}_{h} ] HV [ {Ab}_{2} , {H}_{h} ] )$$

In the above algorithm, HV indicates a lookup into the table of hidden variables (e.g., the hidden logits after they have been transformed into the range (0 . . . 1) using the sigmoid function). The value a can represent a temperature parameter on the outer sigmoid, which can take any suitable value (e.g., 5, or any other suitable value). Note that this embodiment of an algorithm implements two applications of the sigmoid function: 1) when creating HV, the table of hidden variables, and 2) at the outermost operation when computing the competes function.

Embodiments can also implement ensemble learning techniques by combining predictions (e.g., competition scores) from multiple hidden variable models trained on different antibody competition data. For example, each hidden variable model can be trained using competition data for different sets of antibodies (e.g., randomized training sets). A prediction about whether two antibodies would compete can be generated by combining the competition scores (e.g., calculated by dot-product operation, as disclosed above) from several trained hidden variable models. The combined score can be a mean, weighted average, or combination calculated by any other suitable mathematical operation.

In some embodiments, the multiple versions of the hidden variable models are trained using different subsets of the antibody competition training data. For example, within a given subset of training data, a majority of pairwise competition measurements for a group of antibodies is wholly removed. In other words, rather than merely removing random pairwise competition measurements to generate a subset of training data, embodiments selectively remove a majority of pairwise competition data for a group of antibodies. This selective removal of competition data for a group of antibodies within the different subsets of training data accomplishes decorrelated versions of the trained hidden variable models. Decorrelated models achieve better results when they are combined in an ensemble approach.

In some embodiments, while a majority of pairwise competition data for a group of antibodies is removed to generate a subset of training data, some competition data for this group can be maintained. For example, a predetermined set of antibodies from the total set of training data can be designated as persistent antibodies, and the competition data for these persistent antibodies can be maintained across the subsets of training data. In these embodiments, when pairwise competition data for a group of antibodies is selectively removed to generate a subset of training data, the pairwise competition data between the group of antibodies and the persistent antibodies is maintained in the subset of training data. These embodiments can train decorrelated models that each benefit from the training supported by the competition data for the persistent antibodies.

One advantage of this ensemble technique is that the variations (e.g., a calculated variance metric) in the predictions from the individual models in the ensemble provide a gauge of the ensemble model's confidence or certainty in its overall prediction. In addition, these variations also provide an indication of how much the prediction may change if the underlying data distribution is changed. In some embodiments, the ensemble technique can combine pairwise antibody competition predictions from one or more hidden variable models and any other suitable model(s) (e.g., landmark correlation model).

Some embodiments leverage a dot-product operation to generate prediction scores. While some simple dot-product models exist to structure and optimize certain problems in machine learning domains, there are differences between embodiments of the optimization model and some existing models:
While certain natural language processing models, such as word2vec, model a probability distribution over neighboring words, embodiments model antibody competition as a binary event. For example, the shift prior to the outer sigmoid is implemented partly due to this difference, as embodiments aim to saturate the outer sigmoid if possible, whereas this is less desired in word2vec;
Some existing models can use many more hidden variables than embodiments, such as large word embeddings. For example, when the hidden variable model according to some embodiments is compared to natural language processing models, some differences are that: the training data in hidden variable model embodiments is smaller; and hidden variable model embodiments are used to inspect hidden variables to uncover patterns.

In some embodiments, the number of hidden variables and the degree of shift after the dot-product are tuneable hyperparameters of the model(s). For example, experimentally 5-10 hidden variables is enough to represent many of the competition patterns within the data for some data sets, however any other suitable number of hidden variables can be implemented. For ease of interpretation, the hidden variable values are constrained to be within the range of (0 . . . 1) in some embodiments.

In some embodiments, model training is used to calculate the hidden variable values. For example, hidden variable values are derived based on the experimental data from the epitope binning run(s). Example experimental data generated by a run is below:

| Analyte | Ligand | Competes |
|---|---|---|
| Ab1 | Ab2 | Yes |
| Ab1 | Ab3 | No |
| Ab1 | Ab4 | Yes |
| Ab1 | Ab5 | No |
| Ab3 | Ab4 | Yes |
| Ab3 | Ab5 | Yes |
| Ab, | Ab5 | Yes |

Note that the tabular formulation of the experimental data supports concatenation, or vertical stacking, of experimental results from different epitope binning runs. This concatenation is used along with a numerical optimization procedure implemented by some embodiments to achieve joint optimization of the hidden variable values using data from multiple different epitope binning runs. Note that in order for the same hidden variables to have the same meaning for antibodies that were present in different runs, a sufficient set of cross-run antibodies that participated in both epitope binning runs is maintained. To optimally select cross-run antibodies, one or more predictive models can identify a small set of antibodies from a first run that are least correlated with each other in their competition behavior, and those can be selected as the cross-run antibodies to be used in subsequent runs.

Embodiments derive the hidden variables and hidden variable values (e.g., hidden variable affinity scores) using numerical optimization techniques, such as forms of gradient descent, to optimize their values such that model predictions correspond to the actual experimental data according to a loss function. A number of potential algorithms or techniques can be used to accomplish this task. Below is an example optimization procedure according to some embodiments.
0) Start with a single hidden variable, HO, initialized with a hidden logit value of 0.0 for all antibodies, which implies an indeterminate hidden variable value of 0.5.
   Note that this is the point on the sigmoid curve where the gradient is highest, i.e., the sigmoid is the least saturated.
1) Optimize a cross-entropy loss of the model's competition predictions on the training set using an optimization procedure such as Broyden-Fletcher-Goldfarb-Shanno algorithm (“BFGS”) or L-BFGS.
   With just a single hidden variable, this optimization problem may be convex.
   The solution tends to use this single hidden variable to represent and predict the single largest competition group in the dataset.
   Optionally, add a small L2-norm term to the cross-entropy
2) After the optimization converges, add a new hidden variable column for each antibody, again initialized to the intermediate logit value of 0.0, implying a hidden variable value of 0.5 for each antibody.
3) Jointly optimize all the hidden variables for all antibodies to predict the pairwise competition events using the same loss function and optimization procedure from Step 1.
   This optimization problem is no longer convex, but since the first hidden variable has presumably moved away from 0.5 for some antibodies, the symmetry is broken and the optimizer can proceed.
   At this point, the optimizer often picks out the second most prevalent competition group in the dataset.
Steps 2 and 3 can be repeated to incrementally add new hidden variables until the model's prediction performance on a held out validation set converges.
   Note that this optimization procedure is entirely deterministic.

This technique of incrementally adding hidden variables is reminiscent of the idea of boosting from machine learning, where an iterative succession of weak learners are trained, each one correcting the errors made by the models in prior iterations. The above optimization approach differs from boosting, however, in that old and new hidden variables are jointly optimized together, allowing the hidden variables from earlier iterations to be refined once new ones are added.

The approach of incrementally adding hidden variables is also vaguely similar to representing a matrix using lower rank approximations, as is done in Principal Component Analysis (“PCA”). However, the above optimization procedure differs from PCA in that: the matrix reconstruction function contains non-linearities and does not follow the structure of a standard truncated singular value decomposition; and the loss function permits the reconstruction of incomplete matrices with missing values, which is what enables joining together multiple epitope binning runs.

Embodiments of the hidden variable model also differ from natural language processing models, such as word2vec, for example at least due to training differences. Some additional differences are:
Embodiments of the hidden variable model optimize using the entire training set rather than batches. For example, the training set in embodiments is magnitudes smaller than that of most natural language processing applications. Accordingly, the entire training set can be used for each round of gradient computation, and a second-order optimization method, like LBFGS, can be used to help speed convergence and avoid hyperparameter tuning.
A second order optimizer may perform better on embodiments of the hidden variable model topology because of the two layers of sigmoids. If any sigmoid saturates, the gradient signal coming through it can be very weak, and embodiments push the sigmoids towards saturation.
Embodiments of the hidden variable model do not require a large number of hidden variables nor a random initialization for them.
Embodiments of the hidden variable model can incrementally add hidden variables, rather than starting with a fixed size embedding.

Other optimization techniques can also be implemented in some embodiments. For example, another option for optimization can be to begin with a fixed number of hidden variables. In these embodiments, a random initialization of the hidden variables can be implemented to break the problem's symmetry and allow the optimizer to make progress, however this implementation avoids the need to incrementally add hidden variables. Any other suitable optimization techniques can be implemented.

In some embodiments, once a model has been trained, the hidden variable affinity scores can be used to understand competition trends among the antibodies. For example, a novel feature of embodiments, when compared with the previously implemented clustering techniques, is that embodiments permit antibodies to be associated with multiple groups. For example, this can be accomplished by thresholding each antibody's affinity score for the hidden variables at some cutoff value, such as 0.5. In some embodiments, an antibody can have an affinity greater than 0.5 for multiple hidden variables and thus belong to more than one group. This novel group membership permits additional questions about how these groups of antibodies intersect.

Some embodiments can be used to detect a pattern of competition referred to herein as a “competition sandwich.” A competition sandwich includes 3 groups of antibodies with the competition profile illustrated in FIG. 5, namely that:
Group A competes a substantial amount with Group B
Group B competes a substantial amount with Group C
Group A DOES NOT compete a substantial amount with Group C

In this case, groups A and C are the “bread slices”, and group B is the sandwich “filling.” FIG. 5 depicts a graph-based view of the competition sandwich.

In some circumstances, a competition sandwich might indicate a partial adjacency or ordering of epitopes, with one “sandwiched” between the other two. A general way to consider this idea is that it identifies groups within a connectivity graph for which transitivity does not hold. This effect might be interesting in many contexts. As an analogy, consider how a competition sandwich may indicate associations among groups of people, where:
Almost everyone in Group A knows each other, and also knows almost everyone in Group B.
Almost everyone in Group B knows each other, and knows almost everyone in Group C.
Almost everyone in Group C knows each other, but hardly knows anyone in Group A

This may indicate that there are two different/distinct underlying reasons why these groups of people know each other, and for some people (e.g., Group B), both reasons apply. Similarly, competition sandwiches can demonstrate different/distinct competition among antibodies in some situations.

In some embodiments, the derived hidden variables and affinity scores for the hidden variables can represent a model that predicts competition among the antibodies without the need for an explicit experimental run to observe competition. In other words, competition can be predicted, using the derived model, for pairs of antibodies that have not been experimentally tested and observed. Embodiments of the derived model can be considered a forecasting or simulation tool for antibody competition. Accordingly, embodiments improve competition testing among antibodies by improving resource and time efficiency.

In some embodiments, the derived model can also forecast high-fidelity competition dynamics among antibodies. For example, antibodies with different hidden variable affinity scores can indicate different binding mechanisms while antibodies with similar hidden variable affinity scores can indicate similar binding mechanisms. The hidden variable affinity score descriptors represent a more distilled view of competition dynamics when compared to previous approaches that merely associate antibodies to individual bins. Embodiments of the derived model enable the selection of antibodies with diverse hidden variable affinities to achieve a more robust monoclonal antibody discovery and manufacturing process.

In some implementations, antibody competition can be predicted using a landmark antibody correlation model. For example, the landmark antibody correlation model can featurize each antibody in terms of its competition with a set of predetermined landmark antibodies. One way to consider these competition measurements with the predetermined landmark antibodies is as a substitute for the hidden variables in the hidden variable model disclosed herein, however the competition measurements with the predetermined landmark antibodies are not hidden.

Below is an example that illustrates the landmark antibody competition model with reference to a set of landmark antibody competition measurements:

| Landmark antibody competition | D | E | F | G |
|---|---|---|---|---|
| Competes with A as ligand | Yes | No | Yes | No |
| Competes with A as analyte | Yes | No | Yes | No |
| Competes with B as ligand | No | Yes | No | No |
| Competes with B as analyte | No | No | No | No |
| Competes with C as ligand | Yes | No | Yes | No |
| Competes with C as analyte | Yes | No | Yes | No |

In this example, antibodies A, B, and C are the landmark antibodies, and measurements have been taken that represent competition with each of the other four antibodies, D, E, F, and G. For a set of antibodies for which pairwise predictions are desired (e.g., antibodies D, E, F, and G), embodiments of the landmark antibody correlation model utilize previously taken competition measurements against the same set of predetermined landmark antibodies (e.g., A, B, C). However, pairwise competition measurements between these other non-landmark antibodies have not been taken, and therefore these values are unknown (e.g., it is unknown whether antibodies D and E compete with each other).

To predict a likelihood of competition between antibodies D and E, correlation between those two antibodies' columns is calculated. Following the example above, since the correlation between columns D and F is 1.0, because they have the exact same competition profile against the landmark antibodies, a high likelihood is predicted that antibodies D and F will compete with each other. On the other hand, the correlation between antibodies D and E is closer to zero, indicating that they are less likely to compete with each other. Note that the correlation of antibody G with all other antibodies is undefined, since the standard deviation of its column is 0, and the standard deviation of each column appears in the denominator of the correlation coefficient.

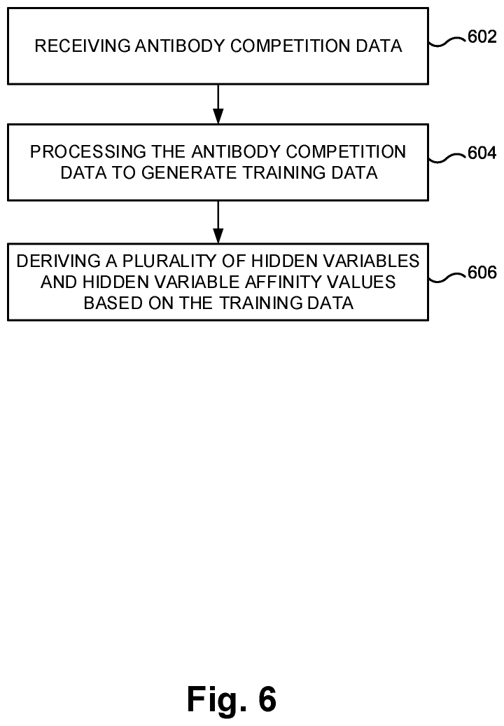

FIG. 6 illustrates a flowchart for deriving hidden variables based on antibody competition data to discover binding patterns according to an example embodiment. In one embodiment, the functionality of FIG. 6 is implemented by software stored in memory or other computer-readable or tangible medium, and executed by a processor. In other embodiments, each functionality may be performed by hardware (e.g., through the use of an application specific integrated circuit (“ASIC”), a programmable gate array (“PGA”), a field programmable gate array (“FPGA”), and the like), or any combination of hardware and software.

At 602, antibody competition data for a plurality of antibodies and an antigen can be received, the antibody competition data including data values indicative of pairwise competition between antibodies. For example, an experimental run (e.g., data generated from surface plasmon resonance (“SPR”) experimental techniques) can generate combinatorial (e.g., pairwise) competition data for a set of antibodies.

In some embodiments, the received antibody competition data includes data from multiple experimental runs, each experimental run generates data values indicative of pairwise competition among a set of antibodies, and the multiple experimental runs generate antibody competition data for different sets of antibodies.

At 604, the antibody competition data can be processed to generate training data. For example, processing the competition data can include data transformations, such as a mathematical transformation to a binary representation for competition. In some embodiments, processing the antibody competition data includes combining the antibody competition data from multiple experimental runs.

At 606, a plurality of hidden variables and affinity scores for the hidden variables can be derived using the training data and an optimization engine, where affinity scores for the hidden variables are derived for each antibody and the hidden variables represent competition factors for the antigen that cause competition among the antibodies. For example, a first hidden variable can represent a first competition factor for the antigen, and a derived affinity score for the first hidden variable associated with a given antibody indicates the given antibody's degree of competition over the first competition factor. In some embodiments, the first competition factor corresponds to an epitope of the antigen that causes competition among the antibodies. In some embodiments, deriving the plurality of hidden variables and the affinity scores for the hidden variables includes deriving affinity scores for the antibodies from different sets of antibodies (e.g., different sets of antibodies involved with different experimental runs).

In some embodiments, the hidden variables are derived by optimizing hidden logit values for the antibodies using pairwise competition data values from the training data, the hidden logit values representing the antibodies' affinity scores for the hidden variables. For example, the antibodies' hidden logit values can be optimized using a loss function, the pairwise competition data values from the training data, and a gradient technique that adjusts the hidden logit values to optimize the loss function.

In some embodiments, the hidden variables and the affinity scores for the hidden variables are derived by initially optimizing the antibodies' hidden logit values for a first hidden variable, and sequentially adding additional hidden variables after the initial optimization of the first hidden variable and jointly optimizing antibodies' hidden logit values for the first hidden variable and each sequentially added additional hidden variable.

In some embodiments, a pairwise competition score prediction for two antibodies can be generated using the hidden logit values optimized for the two antibodies. For example, the received antibody competition data (e.g., processed to generate training) may not include pairwise competition data for the two antibodies. In some embodiments, the pairwise competition score prediction is generated, in part, by performing a dot product operation on the hidden logit values for the two antibodies.

In some embodiments, the derived hidden variables and affinity scores for the hidden variables can represent a model that predicts competition among the antibodies without the need for an explicit experimental run to observe competition. In other words, competition can be predicted, using the derived model, for pairs of antibodies that have not been experimentally tested and observed. Embodiments of the derived model can be considered a forecasting or simulation tool for antibody competition. Accordingly, embodiments improve competition testing among antibodies by improving resource and time efficiency.

In some embodiments, the derived model can also forecast high-fidelity competition dynamics among antibodies. For example, antibodies with different hidden variable affinity scores can indicate different binding mechanisms while antibodies with similar hidden variable affinity scores can indicate similar binding mechanisms. The hidden variable affinity score descriptors represent a more distilled view of competition dynamics when compared to previous approaches that merely associate antibodies to individual bins. Embodiments of the derived model enable the selection of antibodies with diverse hidden variable affinities to achieve a more robust monoclonal antibody discovery and manufacturing process.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

We claim:

1. A method for deriving hidden variables based on antibody competition data to discover binding patterns, the method comprising:
receiving antibody competition data for a plurality of antibodies and an antigen, the antibody competition data comprising data values indicative of pairwise competition between antibodies;
processing the antibody competition data to generate training data; and
deriving, using the training data and an optimization engine, a plurality of hidden variables and affinity scores for the hidden variables, wherein affinity scores for the hidden variables are derived for each antibody and the hidden variables represent competition factors for the antigen that cause competition among the antibodies.

2. The method of claim 1, wherein a first hidden variable represents a first competition factor for the antigen, and a derived affinity score for the first hidden variable associated with a given antibody indicates the given antibody's degree of competition over the first competition factor.

3. The method of claim 2, wherein the first competition factor corresponds to an epitope of the antigen that causes competition among the antibodies.

4. The method of claim 2, wherein the received antibody competition data comprises data from multiple experimental runs, each experimental run generates data values indicative of pairwise competition among a set of antibodies, and the multiple experimental runs generate antibody competition data for different sets of antibodies.

5. The method of claim 4, wherein processing the antibody competition data comprises combining the antibody competition data from the multiple experimental runs.

6. The method of claim 5, wherein deriving the plurality of hidden variables and the affinity scores for the hidden variables comprises deriving affinity scores for the antibodies from the different sets of antibodies.

7. The method of claim 1, wherein the hidden variables are derived by optimizing hidden logit values for the antibodies using pairwise competition data values from the training data, the hidden logit values representing the antibodies' affinity scores for the hidden variables.

8. The method of claim 7, wherein the antibodies' hidden logit values are optimized using a loss function, the pairwise competition data values from the training data, and a gradient technique that adjusts the hidden logit values to optimize the loss function.

9. The method of claim 8, wherein the hidden variables and the affinity scores for the hidden variables are derived by:
initially optimizing the antibodies' hidden logit values for a first hidden variable; and
sequentially adding additional hidden variables after the initial optimization of the first hidden variable and jointly optimizing antibodies' hidden logit values for the first hidden variable and each sequentially added additional hidden variable.

10. The method of claim 7, further comprising:
generating a pairwise competition score prediction for two antibodies using the hidden logit values optimized for the two antibodies.

11. The method of claim 10, wherein the received antibody competition data does not include pairwise competition data for the two antibodies.

12. A system for deriving hidden variables based on antibody competition data to discover binding patterns, the system comprising:
a processor; and
a memory storing instructions for execution by the processor, the instructions configuring the processor to:
receive antibody competition data for a plurality of antibodies and an antigen, the antibody competition data comprising data values indicative of pairwise competition between antibodies;
process the antibody competition data to generate training data; and
derive, using the training data and an optimization engine, a plurality of hidden variables and affinity scores for the hidden variables, wherein affinity scores for the hidden variables are derived for each antibody and the hidden variables represent competition factors for the antigen that cause competition among the antibodies.

13. The system of claim 12, wherein a first hidden variable represents a first competition factor for the antigen, and a derived affinity score for the first hidden variable associated with a given antibody indicates the given antibody's degree of competition over the first competition factor.

14. The system of claim 13, wherein the first competition factor corresponds to an epitope of the antigen that causes competition among the antibodies.

15. The system of claim 13, wherein the received antibody competition data comprises data from multiple experimental runs, each experimental run generates data values indicative of pairwise competition among a set of antibodies, and the multiple experimental runs generate antibody competition data for different sets of antibodies.

16. The system of claim 15, wherein processing the antibody competition data comprises combining the antibody competition data from the multiple experimental runs.

17. The system of claim 16, wherein deriving the plurality of hidden variables and the affinity scores for the hidden variables comprises deriving affinity scores for the antibodies from the different sets of antibodies.

18. The system of claim 12, wherein the hidden variables are derived by optimizing hidden logit values for the antibodies using pairwise competition data values from the training data, the hidden logit values representing the antibodies' affinity scores for the hidden variables.

19. The system of claim 18, wherein the antibodies' hidden logit values are optimized using a loss function, the pairwise competition data values from the training data, and a gradient technique that adjusts the hidden logit values to optimize the loss function.

20. A non-transitory computer readable medium having instructions stored thereon that, when executed by a processor, cause the processor to derive hidden variables based on antibody competition data to discover binding patterns, wherein, when executed, the instructions cause the processor to:
receive antibody competition data for a plurality of antibodies and an antigen, the antibody competition data comprising data values indicative of pairwise competition between antibodies;
process the antibody competition data to generate training data; and
derive, using the training data and an optimization engine, a plurality of hidden variables and affinity scores for the hidden variables, wherein affinity scores for the hidden variables are derived for each antibody and the hidden variables represent competition factors for the antigen that cause competition among the antibodies.
